(19) United States
(12) Patent Application Publication
(10) Pub. No.: US 2020/0129646 A1
(43) Pub. Date: Apr. 30, 2020

(54) Radioactive labeled long-acting peptide targeted pharmaceutical and production method thereof

(71) Applicants: Ming-Hsin Li, Taoyuan (TW); Chih-Hsien Chang, Taoyuan (TW); Su-Jung Chen, Taoyuan (TW); Shih-Ying Lee, Taoyuan (TW); Sheng-Nan Lo, Taoyuan (TW); Ming-Wei Chen, Taoyuan (TW); Yuan-Ruei Huang, Taoyuan (TW); Chun-Fang Feng, Taoyuan (TW); Shih-Wei Lo, Taoyuan (TW); Cheng-Hui Chuang, Taoyuan (TW)

(72) Inventors: Ming-Hsin Li, Taoyuan (TW); Chih-Hsien Chang, Taoyuan (TW); Su-Jung Chen, Taoyuan (TW); Shih-Ying Lee, Taoyuan (TW); Sheng-Nan Lo, Taoyuan (TW); Ming-Wei Chen, Taoyuan (TW); Yuan-Ruei Huang, Taoyuan (TW); Chun-Fang Feng, Taoyuan (TW); Shih-Wei Lo, Taoyuan (TW); Cheng-Hui Chuang, Taoyuan (TW)

(21) Appl. No.: 16/175,859

(22) Filed: Oct. 31, 2018

(51) Int. Cl. A61K 51/08; A61K 51/04; C07B 59/00; A61K 49/00; C07K 7/64

(52) U.S. Cl. CPC A61K 51/088; A61K 51/0482; C07B 59/008; C07B 2200/05; C07K 7/64; A61K 2123/00; A61K 49/0002

(57) ABSTRACT

A radioactive labeled long-acting peptide-targeting pharmaceutical and production method, in which the peptide targeted pharmaceutical is firstly dissolved in a solution, followed by labeling the radioactive at a high temperature, and the dosage of the pharmaceutical with radioactive labeling is expected to be reduced and labeling efficiency is improved, and no further purification by filtration is required, which shortens the preparation process and reduces personnel exposure in the working environment. The radioactive labeled long-acting peptide-targeting pharmaceutical can increase the specific binding capacity of tumors and reduce the non-specific accumulation in normal tissues. It can be applied to the field of tumor and nuclear medicine for diagnosis and treatment of tumors and/or tumor metastases with efficacy and precision treatment.

### BACKGROUND OF THE INVENTION

### 1. Field of the Invention

The invention relates to a radioactive labeled long-acting targeted peptide pharmaceutical and a production method thereof, in particular to a targeted peptide pharmaceutical produced with improved labeling efficiency and minimum dosage required that reduces preparation time and cost.

### 2. Description of Related Art

According to the Global Cancer Report 2014 published by the World Health Organization (WHO), global cancer cases are growing rapidly from 14 million in 2012 to 19 million in 2025, and will reach 24 million by 2035. That is an increase of more than 70% in 23 years. At present, the development of targeted peptide pharmaceuticals has been a trend in decade. Tumor cells grow to 1-2 mm while migrate and grow due to their new blood vessels, and integrin acts as an influence on cell attachment, migration, growth and differentiation, and angiogenesis are highly expressed on inflammation and tumor endothelial cells, also, αvβ3 or αvβ5 plays an important role in breast cancer angiogenesis.

According to the Global Cancer Report 2014 published by the World Health Organization (WHO), global cancer cases are growing rapidly, from 14 million in 2012 to 19 million in 2025, and will reach 24 million by 2035. That is, an increase of more than 70% in 23 years. At present, the development of targeted peptide pharmaceuticals has been a trend in the past decade.

Tumor cells grow to 1-2 mm and migrate and grow due to their new blood vessels. Integrin acts as an influence on cell attachment, migration, growth and differentiation and angiogenesis is highly expressed in inflammation and tumor endothelial cells, in which, αvβ3 or αvβ5 also play an important role in breast cancer angiogenesis.

Therefore, the RGD peptide is used as an integrin-ligand interaction inhibitor, and the circular RGD peptide preferentially binds to integrin αvβ3 as a tumor cell targeting probe. When the peptide is labeled with a long half-life and a short half-life of radioisotopes, the molecular imaging can be used as a diagnostic and therapeutic agent for tumors.

Since members of the integrin family play an important role in the regulation of cell activation, migration, proliferation, survival, and differentiation, integrin αvβ3 has been found in osteoclasts and invasive tumors, such as advanced glioblastoma, breast and prostate tumors, malignant melanoma, and ovarian cancer, can be highly expressed, and clinical trials have also begun using radiolabeled RGD-peptides for integrin αvβ3, such as F-18-labeled RGD, for non-invasive breast cancer testing and staging. RGD peptides and labeling radionuclides, such as I-131, In-111, Tc-99m, Ga-68, F-18, etc. are used for visualizing tumor sites, such as some F-18-galactose RGD, F-18-RGD-K5, Ga-68-NOTA-RGD, Ga-68-NOTA-(RGD)2, F-18-(RGD)2 and F-18-Alfatide. At present, the development of clinical pharmaceutical RGD pharmaceuticals is mainly RGD or c(RGDfV) or c(RGDfK), and most of them are diagnostic agents.

Since members of the integrin family play an important role in the regulation of cell activation, migration, proliferation, survival and differentiation, integrin αvβ3 has been found highly expressed in osteoclasts and invasive tumors, such as advanced glioblastoma, mammary gland and prostate tumors, malignant melanoma, and ovarian cancer; clinical use of radiolabeled RGD-peptides for integrin αvβ3, such as F-18-labeled RGD, has also begun to be used for non-invasive breast cancer detection and staging. Contains RGD peptides and uses labeled radionuclides, such as I-131, In-111, Tc-99m, Ga-68, F-18, etc. for imaging tumor sites, such as some F-18-galactose, RGD, F-18-RGD-K5, Ga-68-NOTA-RGD, Ga-68-NOTA-(RGD)2, F-18-(RGD)2 and F-18-Alfatide. At present, the development of clinical pharmaceutical RGD pharmaceuticals is mainly RGD. c(RGDfV), or c(RGDfK), and most of them are diagnostic agents.

However, the peptide pharmaceutical is good in water solubility, and the amount of tumor accumulation in the actual application is not as high as that of the protein pharmaceutical, and is rapidly metabolized in the body. In the process of commercialization, it is still necessary to overcome the T/M ratio during radiography, the decrease of tumor accumulation amount with time, and the rapid elimination of small molecule peptide pharmaceuticals. In the prior arts WO2016209795A1 and CN 104650217A, National Institutes of Health revealed the modified Evans Blue (EB) as the technology for the development of RGD-related pharmaceuticals, but it is also difficult to effectively overcome problem of radiation decay in storage and transport.

### SUMMARY OF THE INVENTION

The main object of the present invention is to provide a radioactive long-acting targeted peptide pharmaceutical DOTA-EB-cRGDfK (1,4,7,10-tetraazacyclododecane-N,N′,N″,N″′-tetraacetic Acid, DOTA), which is based on the highly specific tumor and the angiogenesis-identifying cyclic RGD peptide pharmaceutical cRGDfK, and develop long-acting target nuclear medicine for systemic transfer of integrin αvβ3 tumors, such as breast cancer etc., for diagnosis and treatment; and the main mechanism and design are as follows:

1) cyclic RGD derivatives: The cyclic peptide cRGDfK is highly specifically integrated with neovascularization and highly expressed integrin αvβ3 on some tumors.

2) the cyclic RGD is connected with a molecule which can be highly affined with albumin in the blood, which can prolong the circulation time of the peptide pharmaceutical in the body, and finally achieve goal of the specific binding amount of the tumor and reduce the non-specificity of the normal tissue in accumulation.

3) bonding a metal chelating agent (DOTA) and labeling radioisotopes with In-111, Lu-177, or Ga-68.

The advantages of the above-mentioned radioactive peptide pharmaceuticals. for example, breast cancer, can accurately assess the distribution and size of tumors in the body, mainly for systemic metastatic breast cancer that cannot be accurately diagnosed by surgery, ultrasound, tomography and reduce the probability of misdiagnosis.

Another object of the present invention is to provide a radiation-labeled long-acting targeted peptide pharmaceutical, which is designed in a kit to produce a long-acting targeted peptide pharmaceutical for the whole operation. It is extremely convenient, and it can be safely stored and transported without being affected by radiation half-time. It can be of more than 90% labeling efficiency being tested by Radio-HPLC and ITLC, and it does not need to be purified by column.

Still another object of the present invention is to provide a radioactive labeled long-acting targeted peptide pharmaceutical which can be combined with molecular imaging medical technology to develop diagnosis and treatment techniques, improving the diagnosis accuracy and cure rate of tumors which can be diagnosed and treated at earlier stage, and reducing cost.

### BRIEF DESCRIPTION OF THE DRAWINGS

FIG. 1 is a chemical structural diagram of the DOTA-EB-cRGDfK of the present invention.

FIG. 2 is a graph (I) showing the effect of the DOTA-EB-cRGDfK of the present invention.

FIG. 3 is a graph (2) showing the effect of the DOTA-EB-cRGDfK of the present invention.

FIG. 4 is a graph showing the pharmacokinetic profile of 111In-DOTA-EB-cRGDfK of the present invention in U-87 MG tumor animal model.

FIG. 5 is a statistical diagram showing the distribution of tumor pharmaceutical in organs of U-87 MG of 111In-DOTA-EB-cRGDfK of the present invention.

FIG. 6 is a graph showing the biodistribution of the tumor pharmaceutical of U-87 MG of the present invention by 111In-DOTA-EB-cRGDfK.

FIG. 7 is a flow chart showing the method of using the kit of 111In-DOTA-EB-cRGDfK of the present invention.

### DETAILED DESCRIPTION OF THE PREFERRED EMBODIMENTS

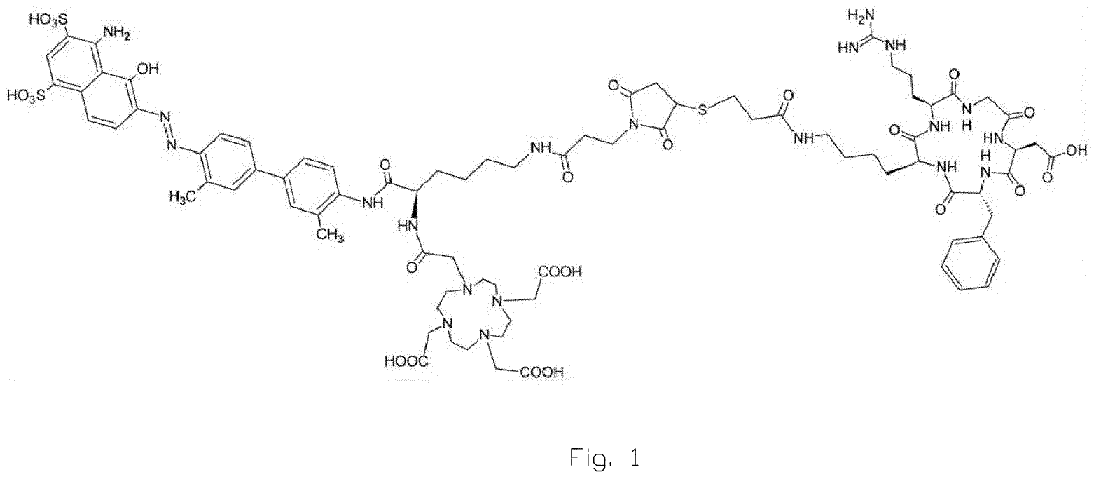

The chemical structure of DOTA-EB-cRGDfK of the present invention is shown In FIG. 1, The radioactive labeled DOTA-EB-cRGDfK is prepared by adding 30 μg of DOTA-EB-cRGDfK peptide containing 20 μg/μL in DMSO solution, adding a radiation solution containing ¹¹¹In or ⁶⁸Ga or ¹⁷⁷Lu), and adding 1M NaOAc solution to make the volume of 300 μL, the pH of the solution is 6-7 after mixing evenly. The radiation solution is placed in a 95° C. thermal reactor for 15 minutes and 1 μL was taken for ITLC analysis after cooling. The labeling efficiency showed greater than 90%. The radiation solution was taken for Radio-HPLC analysis at 20-30 μCi and the main signal showed a retention time of 7 minute.

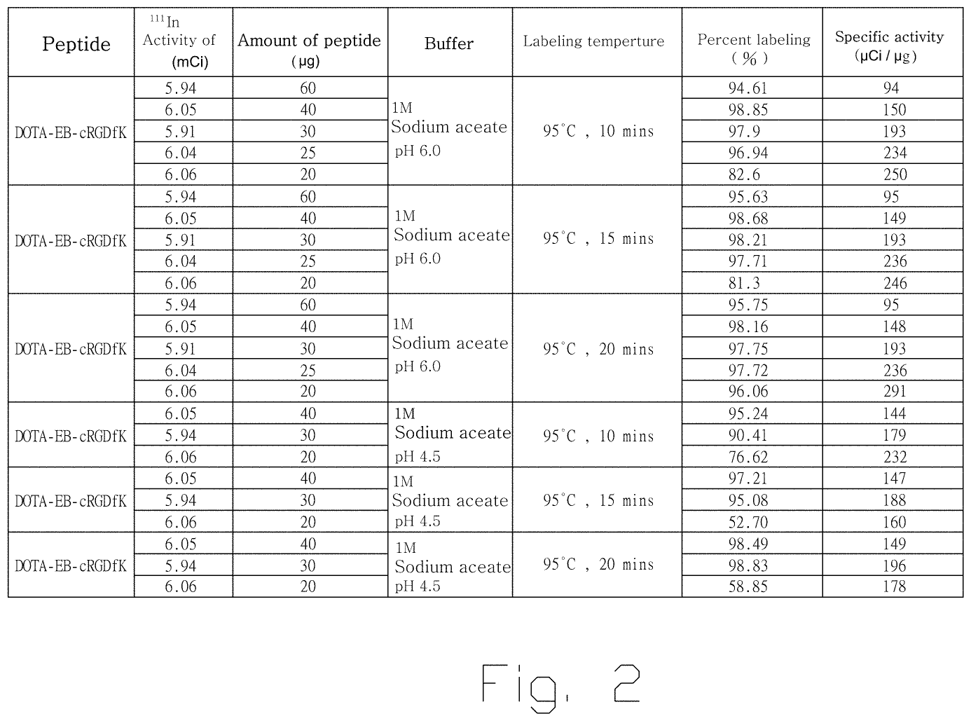

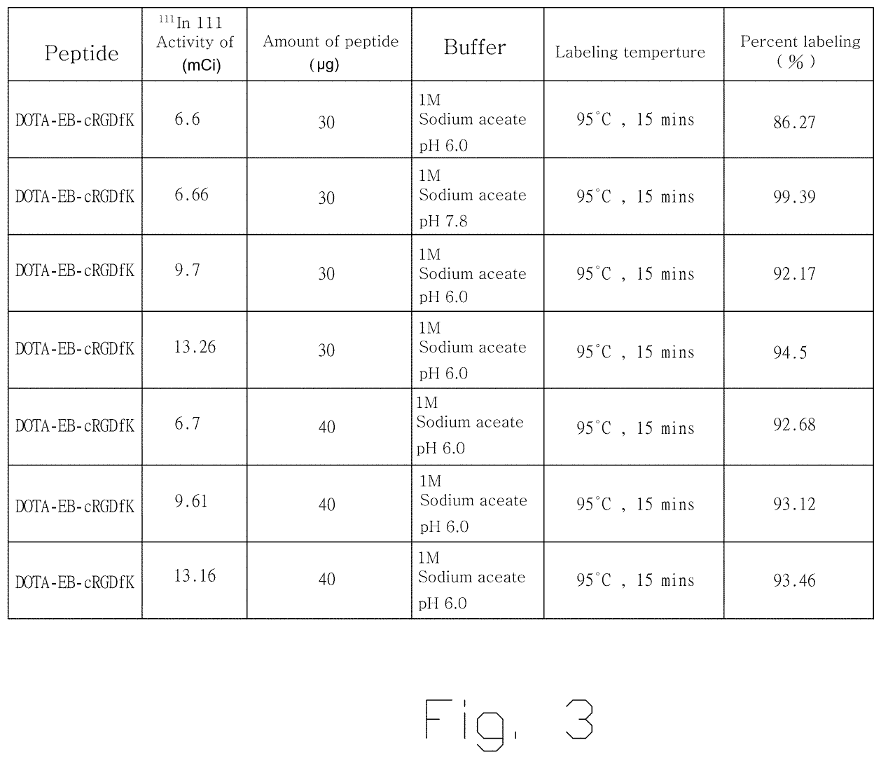

Please refer to FIG. 2, which is a diagram showing the effect of concentration and pH of the radioactive labeled DOTA-EB-cRGDfK of the present invention, and FIG. 3, which shows the effect of radioactive labeled DOTA-EB-cRGDfK of the present invention, and the activity and concentration statistics table indicate the result of the radioactive labeled DOTA-EB-cRGDfK under conditional test as follows:

1) The content of DOTA-EB-cRGDfK is 40 to 60 μg, the In¹¹¹ activity is 6mCi, and the reaction is carried out at 95° C. for 10 to 20 minutes at pH 4.5-6, and the labeling efficiency is greater than 90%.

2) The content of DOTA-EB-cRGDfK is 40 to 60 μg, the In¹¹¹ activity is 6 to 13mCi, and the reaction is carried out at 95° C. for 15 minutes at pH6, and the labeling efficiency is greater than 90%.

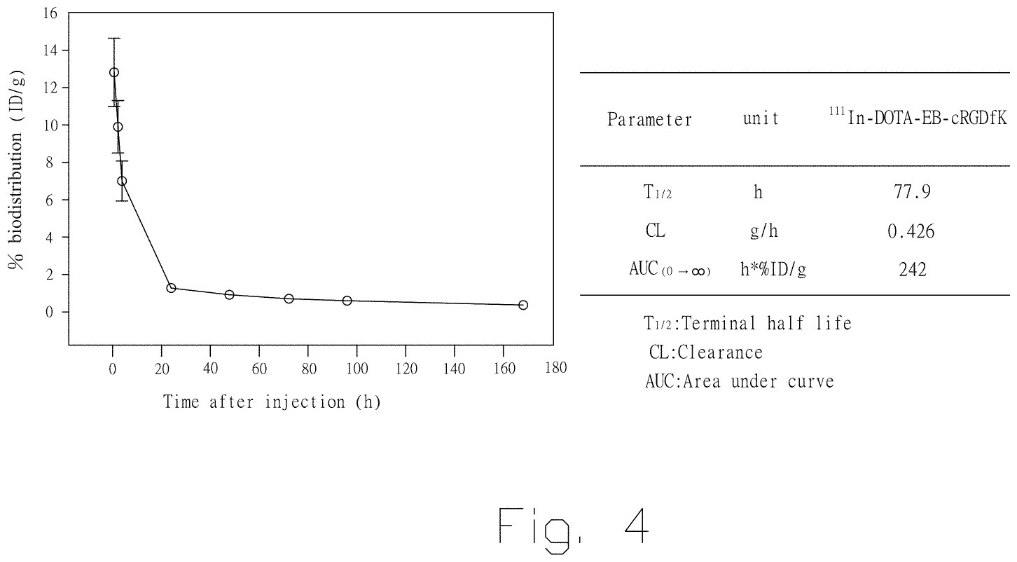

Referring to FIG. 4, it can be seen that the above 111In-DOTA-EB-cRGDfK of the present invention is administered in the U-87 MG tumor animal model for pharmacokinetic test, and the ¹¹¹In-DOTA-EB-cRGDfK is administered via the tail vein at different times and the blood was taken for pharmacokinetic analysis at different times. As a result, the pharmaceutical half-life (T1/2) was 77.9 hours, the clearance (CL) was 0.426 g/h, and the area under curve (AUC) was 242 h*% ID/g.

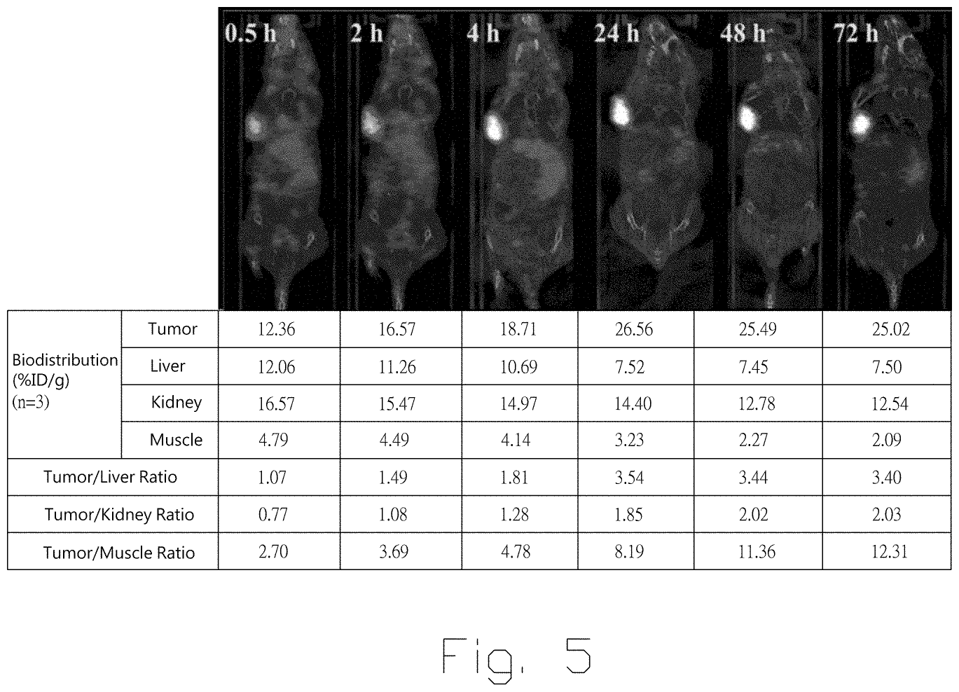

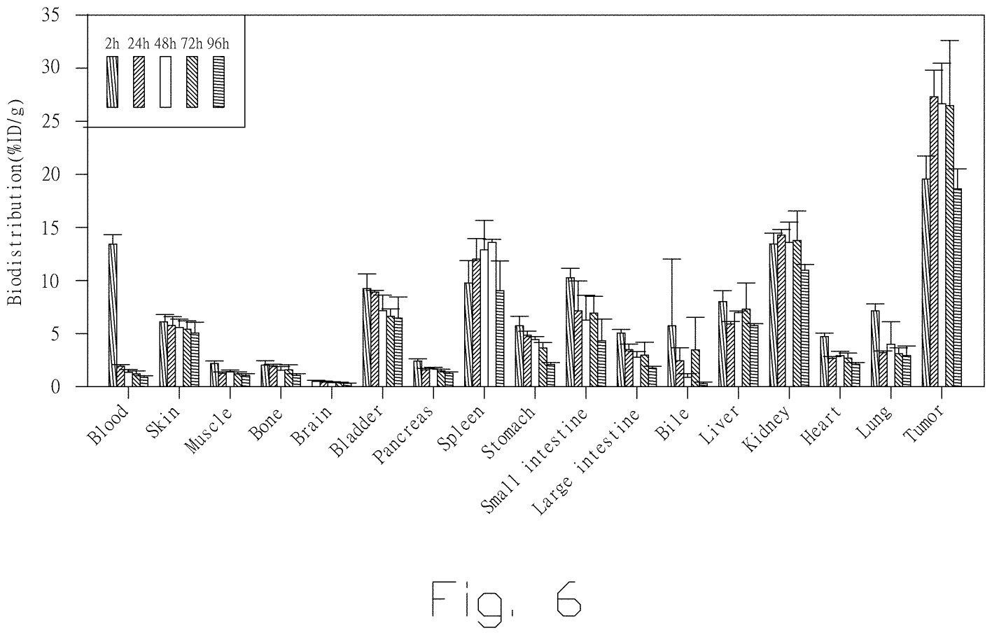

Referring to FIGS. 5 and 6, it can be seen that the above ¹¹¹In-DOTA-EB-cRGDfK of the present invention is administered in the U-87 MG tumor pharmaceutical for biodistribution test, and the ¹¹¹In-DOTA-EB-cRGDfK is administered via the tail vein at different times, and the tissue/organ of the sacrificial animal is taken for samples to perform a radiation dosimeter reading and converts it into organism distribution data (% ID/g). The results showed that the ¹¹¹In-DOTA-EB-cRGDfK had a significant accumulation in U-87 MG tumors up to approximately 25% ID/g and retained for at least 96 hours.

In the practical application, the above-mentioned radioactive labeled DOTA-EB-cRGDfK of the present invention can be prepared into a DOTA-EB-cRGDfK cryostat bottle, which is convenient for long-term storage and transportation, and can improve the convenience of use; DOTA- The preparation method of EB-cRGDfK cryostat bottle is as follows:

adding 4-5 μL of DOTA-EB-cRGDfK/DMSO solution with concentration 20 μg/μL to 333 μL in volume of 3M sodium acetate solution, adding injection water to obtain a volume of 1000 μL with pH 7, and freeze-drying at −25° C. for 96 hours, refilling with nitrogen for about 1 minute, and sealing the cap with a plastic soft plug and aluminum cap.

In practical application, the DOTA-EB-cRGDfK cryostat bottle can be combined with a solution bottle, a filtering device and an adsorption device to form a kit suitable for production of the radioactive labeled DOTA-EB-cRGDfK of In¹¹¹ and Ga⁶⁸. The solution bottle has a content of 3 mL of hydrochloric acid or acetic acid of pH 3 for use as a conditioning agent and pH adjustment.

The components of the DOTA-EB-cRGDfK cryostat bottle include:

1) a content of 60-120 μg, preferably 100 μg of DOTA-EB-cRGDfK peptide;

2) dimethyl sulfoxide DMSO having a content of 4-10 μL;

3) sodium acetate in an amount of 80-100 mg.

The adsorption device has an adsorption column, the composition of which has two layers, and the upper layer is an enhanced anion exchange resin, and the composition thereof includes Dowex 1×8-200, 100-200 mesh, 8% cross-linking, the dosage is 0.5 g to 2 g, and the lower layer is titanium dioxide particles with a size 75-300 μm and the amount is zero or 0.1 g to 1 g, and its composition includes rutile or anatase filled in a Biorad column (731-1550). The filtration device is a 0.22 μm PVDF (polyvinylidene fluoride) filter.

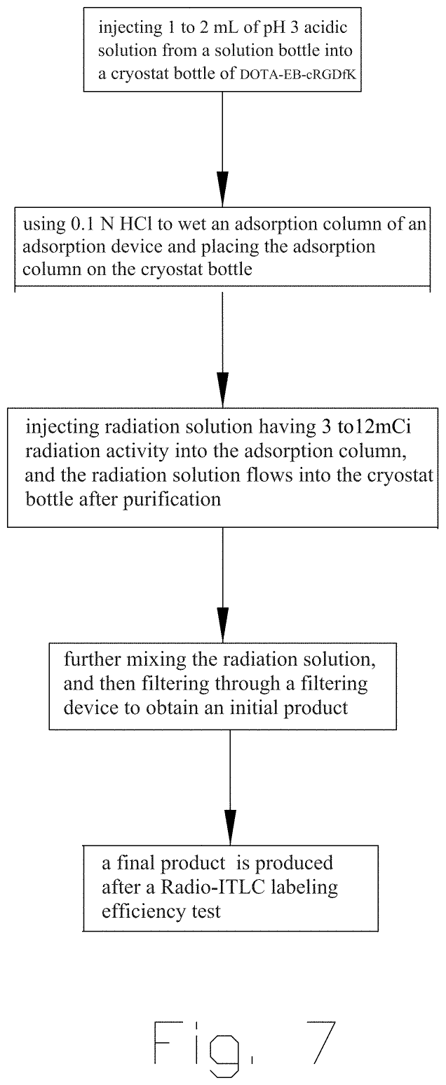

Referring to FIG. 7, it can be seen that the above-mentioned ¹¹¹In-DOTA-EB-cRGDfK or ⁶⁸Ga-DOTA-EB-cRGDfK or ¹⁷⁷Lu-DOTA-EB-cRGDfK kit of the present invention has the following steps:

injecting 1 to 2 mL of pH 3 acidic solution from a solution bottle into a cryostat bottle of DOTA-EB-cRGDfK;

using 0.1 N HCl to wet an adsorption column of an adsorption device and placing the adsorption column on the cryostat bottle;

injecting radiation solution of ⁶⁸GaCl₃ or ¹¹¹InCl₃ or ¹⁷⁷LuCl₃ having 3 to 12mCi radiation activity into the adsorption column, and the radiation solution flows into the cryostat bottle after purification;

heating a chemical reaction bottle from 85 to 95° C., preferably 95° C., for 10 to 30 minutes for further mixing the radiation solution, and then filtering through a filtering device to obtain an initial product;

a final product of the radioactive labeled ¹¹¹In-DOTA-EB-cRGDfK or ⁶⁸Ga-DOTA-EB-cRGDfK or ¹⁷⁷Lu-DOTA-EB-cRGDfK is produced after a Radio-ITLC labeling efficiency test.

The above DOTA-EB-cRGDfK cryostat was subjected to the In-111 labeling test, and the labeling efficiency and radiochemical purity test were carried out by ITLC and Radio-HPLC, and the 111In-DOTA-EB-cRGDfK labeling efficiency and radiochemical purity were greater than 90%, its cryostat bottle can still reach radiochemical purity and labeling efficiency of greater than 90% after maintaining at −25° C. for 1 month.

The above-mentioned radiation labeled long-acting targeted peptide pharmaceutical of the present invention has the following advantages:

1) Increasing the amount of pharmaceutical specific to the tumor, and reducing the amount of pharmaceutical accumulation in the non-tumor tissue.

2) Improving shortcomings of rapid elimination rate of peptide pharmaceuticals.

3) The amount of salt composition is small to litigate the burden on the body.

4) To be used for diagnosis and treatment.

5) The preparation process is short and personnel exposure is reduced.

6) Radiation species, such as, ⁶⁸Ga, ¹¹¹In, and ¹⁷⁷Lu that can be used for diagnosis and treatment.

In addition, the combination of its pharmaceutical and albumin in the blood can prolong the circulation time of the peptide pharmaceutical in the body, increase the specific binding amount of the tumor, and reduce the non-specific accumulation of normal tissues to meet the development trend of precision medicine.

In practical applications, the ¹⁷⁷Lu/⁹⁰Y-DOTA-EB-cRGDfK for therapeutic use can be further developed in the future to achieve the goal of personalized medicine by diagnosis and screening first, and followed by precise treatment. The advantage is to accurately assess the distribution of breast cancer in the body and the size of the tumor, mainly for the surgical operation, ultrasound, tomography and those other methods, which can't be used accurately diagnose the systemic metastatic Integrin αvβ3 tumor and thus increasing the diagnostic accuracy.

What is claimed is:

1. A radioactive labeled long-acting peptide targeted pharmaceutical consisting of radiolabeled DOTA-EB-cRGDfK peptide derivative, including a chelating agent, an Evans blue (EB), a cyclic peptide cRGDfK derivative; a chemical structure of DOTA-EB-cRGDfK peptide derivative is shown below:

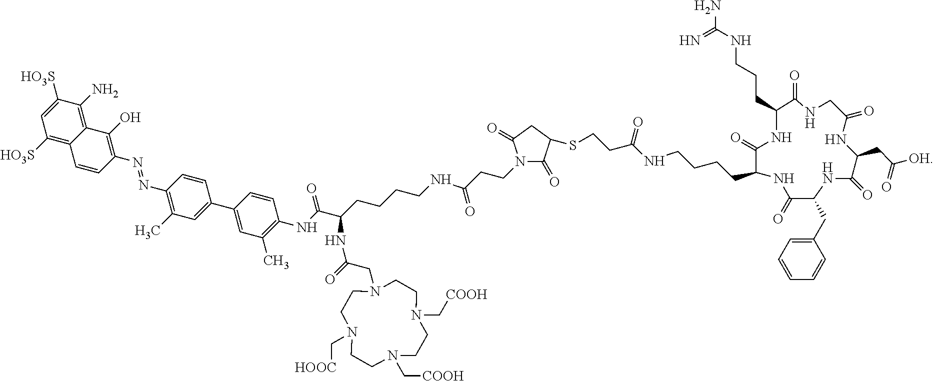 [SMILES: Cc1cc(-c2ccc(NC(=O)[C@@H](CCCCNC(=O)CCN3C(=O)CC(SCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C3=O)NC(=O)CN3CCN(CC(=O)O)CCN(CC(=O)O)CCN(CC(=O)O)CC3)c(C)c2)ccc1/N=N/c1ccc2c(S(=O)(=O)O)cc(S(=O)(=O)O)c(N)c2c1O]

2. The radioactive labeled long-acting targeted peptide pharmaceutical according to claim 1, wherein the chelating agent comprises a metal chelate compound for labeling with radioisotope.

3. The radioactive labeled long-acting targeted peptide pharmaceutical according to claim 1, wherein the radioisotope is selected from one of radioactive isotopes ⁶⁸Ga, ¹¹¹In, and ¹⁷⁷Lu.

4. A kit of radioactive labeled long-acting targeted peptide pharmaceutical, comprising a cryostat bottle containing DOTA-EB-cRGDfK, a solution bottle, a filtering device, an adsorption device, is provided for containing a radioactive labeled long-acting targeted peptide pharmaceutical.

5. The kit of radioactive labeled long-acting targeted peptide pharmaceutical according to claim 4, wherein the solution bottle is provided with an acidic solution of pH 3.

6. The kit of radioactive labeled long-acting targeted peptide pharmaceutical according to claim 5, wherein the acidic solution in the solution bottle is one selected from the group consisting of hydrochloric acid, acetic acid and citric acid.

7. The kit of radioactive long-acting targeted peptide pharmaceutical according to claim 4, wherein the filter device is a 0.22 μm PVDF filter.

8. The kit of radioactive long-acting targeted peptide pharmaceutical according to claim 4, wherein the DOTA-EB-cRGDfK contained cryostat bottle having main components of a single dose after mixing includes: DOTA-EB-cRGDfK peptide 60 to 120 μg, dimethyl sulfoxide DMSO 2 to 10 μL, and sodium acetate 80 to 100 mg.

9. The kit of radioactive long-acting targeted peptide pharmaceutical according to claim 4, wherein the adsorption device is a two-layer adsorption column, including the upper layer of the adsorption column of an enhanced anion exchange resin and the composition of Dowex 1×8-200, 100-200 mesh, 8% cross-linking and 0.5 g˜2 g dosage, and including the lower layer of the adsorption column includes titanium dioxide particles with size 75-300 μm and zero dosage and the composition of rutile or anatase filled in a Biorad column (731-1550) column.

10. A method for using the kit of radiation-labeled long-acting targeted peptide pharmaceutical of claim 4, comprising following steps:
injecting 1 to 2 mL of pH 3 acidic solution from a solution bottle into a cryostat bottle of DOTA-EB-cRGDfK;
using 0.1 N HCl to wet an adsorption column of an adsorption device and placing the adsorption column on the cryostat bottle;
injecting a radiation solution into the adsorption column, and the radiation solution flows into the cryostat bottle after purification;
heating a chemical reaction bottle in a range from 85 to 95° C. for 10 to 30 minutes for further mixing the radiation solution, and then filtering through a filtering device to obtain an initial product;
a final product of the radioactive labeled DOTA-EB-cRGDfK is produced after a Radio-ITLC labeling efficiency test.

11. The method for using a radioactive labeled long-acting targeted peptide pharmaceutical according to claim 10, wherein the radiation solution is ¹¹¹InCl₃, ⁶⁸GaCl₃, or ¹⁷⁷LuCl₃ with solution activity 3 to 12 mCi and a volume 10 to 200 μL.

12. The method for using a radioactive labeled long-acting targeted peptide pharmaceutical according to claim 10, wherein the final product of the radioactive labeled DOTA-EB-cRGDfK is ¹¹¹In-DOTA-EB-cRGDfK, ⁶⁸Ga-DOTA-EB-cRGDfK or ¹⁷⁷Lu-DOTA-EB-cRGDfK.
